(19) United States
(12) Patent Application Publication
(10) Pub. No.: US 2017/0303991 A1
(43) Pub. Date: Oct. 26, 2017

(54) Monitoring Electrolysis

(71) Applicant: The Regents of the University of California, Oakland, CA (US)

(72) Inventors: Boris Rubinsky, El Cerrito, CA (US); Arie Meir, Oakland, CA (US); Mohammad Hjouj, Berkeley, CA (US)

(73) Assignee: The Regents of the University of California, Oakland, CA (US)

(21) Appl. No.: 15/504,522

(22) PCT Filed: Aug. 27, 2015
(86) PCT No.: PCT/US15/47219

§ 371 Date: Feb. 16, 2017

Related U.S. Application Data
(60) Provisional application No. 62/043,049, filed on Aug. 28, 2014.

(51) Int. Cl. A61B 18/14; A61B 5/145; G01R 33/48; A61F 9/007; A61B 5/053; A61B 5/055; A61B 18/00

(52) U.S. Cl. CPC A61B 18/1402; A61B 5/055; A61B 5/14539; G01R 33/4808; A61F 9/007; G01R 33/4804; A61B 5/0536; A61B 2018/00577; A61B 2018/00642; A61B 2018/00982; A61B 2018/1467; A61B 2018/00875

(57) ABSTRACT

Methods and compositions are provided for monitoring and optimizing electrolysis, for example, tissue electrolysis. Aspects of the methods include monitoring electrolysis of a tissue in a subject using an imaging technique or a measurement technique, e.g., a bulk spectroscopic measurement technique. Imaging techniques of interest include electrical impedance-based tomography and magnetic electrical impedance tomography. Electrical impedance-based imaging methods include imaging the electrical impedance of a tissue of the subject undergoing electrolysis, and monitoring the electrolysis based on one or more electrical impedance images of the tissue. Another modality to monitor electrolysis is by magnetic resonance imaging (MRI)-based methods which include imaging pH changes in a tissue of the subject undergoing electrolysis by magnetic resonance imaging, and monitoring the electrolysis based on one or more magnetic resonance images of the pH changes in the tissue. Measurement techniques of interest include bulk measurements of electrical properties and their changes with electrolysis or bulk changes in magnetic resonance readings and their changes with electrolysis. Devices and systems thereof that find use in practicing the methods are also provided.

### INTRODUCTION

The manipulation of tissue by electrolysis has an increasing role in the treatment of many diseases and conditions. For example, electrolysis may be used in the ablation of tissue. Tissue ablation with minimally invasive surgery finds use in the treatment of solid neoplasms. A variety of biophysical and biochemical processes are used for the purpose of tissue ablation, including, for example thermal ablation with heating, cooling or freezing, electroporation, injection of chemical agents, photodynamic effects, sonoporation effects and many others. Electrolysis provides a safe and effective method for ablating tissue limited only by a lack of an effective means to monitor the extent of tissue ablation in the body.

### SUMMARY OF THE INVENTION

Methods and compositions are provided for monitoring and optimizing electrolysis, for example, tissue electrolysis. Aspects of the methods include monitoring electrolysis of a tissue in a subject using an imaging technique or a measurement technique, e.g., a bulk spectroscopic measurement technique. Imaging techniques of interest include electrical impedance-based tomography and magnetic electrical impedance tomography. Electrical impedance-based imaging methods include imaging the electrical impedance of a tissue of the subject undergoing electrolysis, and monitoring the electrolysis based on one or more electrical impedance images of the tissue. Another modality to monitor electrolysis is by magnetic resonance imaging (MRI)-based methods which include imaging pH changes in a tissue of the subject undergoing electrolysis by magnetic resonance imaging, and monitoring the electrolysis based on one or more magnetic resonance images of the pH changes in the tissue. Measurement techniques of interest include bulk measurements of electrical properties and their changes with electrolysis or bulk changes in magnetic resonance readings and their changes with electrolysis. Devices and systems thereof that find use in practicing the methods are also provided.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. Electrical Impedance Tomography System (a) EIT System Schematic (reproduced from [29]) (b) Schematic of Experimental EIT Chamber for Electrolysis Experiments (c) EIT Chamber With Central Electrolysis Electrode in Our Experimental Setup.

FIG. 2. Anode Centered Electrolysis Experiment. EIT images: (a) after 1 minute, (b) after 3 minutes, (c) after 19 minutes. Optical images: (1) after 1 minute, (2) after 3 minutes, (3) after 19 minutes, (4) after 19 minutes with increased contrast.

FIG. 3. Cathode Centered Electrolysis Experiment. EIT images: (a) after 2 minute, (b) after 6 minutes, (c) after 36 minutes. Optical images: (1) after 2 minute, (2) after 6 minutes, (3) after 36 minutes, (4) after 36 minutes with increased contrast

FIG. 4. Two Electrodes Electrolysis Experiment. EIT images: (a) after 1 minute, (b) after 3 minutes, (c) after 6 minutes, (d) after 12 minutes. Optical images: (1) after 1 minute, (2) after 3 minutes, (3) after 6 minutes, (4) after 12 minutes.

FIG. 5. Bacterial Viability Experiment. EIT images: (a) After 15 minutes, (b) After 30 minutes, (c) After 45 minutes. (d) Optical image of growth patterns after 24 hour incubation.

FIG. 6. Experimental setup and control images for different experimental modalities for monitoring electrolysis by magnetic resonance imaging (MRI). a) Experimental setup; b) T1 control study; c) T2 control study; d) PD control study; e) Phenolphthalein 1%; f) Hagen wide range pH indicator dye; g) E. coli control study.

FIG. 7. Comparative MRI imaging results. Each row corresponds to a sequence modality (Top to bottom: T1,T2, PD). Each column corresponds to a stimulation voltage (Left to right: 3V, 6V, 9V).

FIG. 8. Comparative pH dyes and bacterial viability results. Each row corresponds to a control modality. Top to bottom: Phenolphthalein 1% pH indicator; Hagen pH indicator; E. coli bacterial viability. Each column corresponds to a stimulation voltage. Left to right: 3V, 6V, 9V.

FIG. 9. Comparative pH dyes and bacterial viability results. Each row corresponds to a control modality. Top to bottom: Phenolphthalein 1% pH indicator; Hagen pH indicator; E. coli bacterial viability. Each column corresponds to a stimulation voltage. Left to right: 3V, 6V, 9V.

### DETAILED DESCRIPTION OF THE INVENTION

Methods and compositions are provided for monitoring and optimizing electrolysis, for example, tissue electrolysis. The monitoring is based on the concept that electromagnetic properties of materials change with exposure to an electrolytic process, and that the magnetic resonance properties of materials change with exposure to an electrolytic process. Aspects of the methods include imaging the electrical impedance of a material undergoing electrolysis, and monitoring or optimizing the electrolytic process based on the electrical impedance images. In addition, devices and systems thereof that find use in practicing the subject methods are provided. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods

In some aspects of the present disclosure, methods are provided for monitoring and optimizing electrolysis. As used herein, electrolysis refers to the passage of an electric current through a material from a first electrode having a first polarity to a second electrode having a second polarity, through the migration of charged ions within the material between the first and second electrodes. Electrolysis is used for a variety of purposes, including the destruction of biological (e.g., pathological) tissue, the promotion of inflammatory processes in tissue, the extraction of metals from ores, the cleaning of archaeological artifacts, and the coating of materials with thin layers of metal (electroplating). While the detailed description herein is focused on monitoring and optimizing tissue electrolysis, it is envisioned that the subject methods, devices and systems will find use in monitoring and optimizing any electrolysis process.

A summarized above, according to certain embodiments, the electrolysis process (e.g. the onset of electrolysis, the extent or progression of electrolysis, the cessation of electrolysis, etc.) is detected by measuring and by imaging the electrical impedance of the material. In certain aspects, such methods include imaging the electrical impedance of a tissue of the subject undergoing electrolysis, and monitoring the electrolysis based on one or more electrical impedance images of the tissue.

As used herein, “electrical impedance” refers to the degree to which an electrical circuit resists electrical-current flow when voltage is impressed across its terminals. Put another way, electrical impedance is a measurement of the conductivity and permittivity of a given material. Impedance expressed in OHMS is the ratio of the voltage impressed across a pair of terminals to the current flow between those terminals. In direct-current (DC) circuits, impedance corresponds to resistance. In alternating current (AC) circuits, impedance is a function of resistance, inductance, and capacitance. Inductors and capacitors build up voltages that oppose the flow of current. This opposition is referred to as reactance, and must be combined with resistance to define the impedance. The resistance produced by inductance is proportional to the frequency of the alternating current, whereas the reactance produced by capacitance is inversely proportioned to the frequency.

A number of techniques have been developed that generate images of the electrical impedance of a material, any of which may be employed in the subject methods. For example, the material may be imaged using electrical impedance tomography when contact electrodes are used and magnetic impedance tomography when non-contact electromagnetic coils are used. As used herein, electrical impedance tomography (“EIT”, also referred to as electrical impedance imaging, applied potential tomography (APT), and conductivity imaging) refers to an imaging technique that relies on differences in bio-electrical properties within the target material, e.g. a biological tissue, to characterize different regions within it and subsequently output an image correlating to such characterization.

Generally speaking, an EIT image is generated by placing a series of electrodes in a predetermined configuration in electrical contact (e.g., galvanically coupled) with the target material to be imaged, e.g. biological tissue. A low level electrical sinusoidal current is injected through one or more of the electrodes and a resulting voltage is measured at the remaining electrodes. This process may be repeated using different input, or drive, electrodes, and electrical currents of different frequencies. By comparing the various input currents with their corresponding resulting voltages, a map of the electrical impedance characteristics of the interior regions of the material being studied, e.g. a biological tissue, can be imaged. Note that it is also possible to map the impedance characteristics of regions of a material by imposing a voltage and measuring a resulting current, or by injecting and measuring combinations of voltages and currents. By correlating the impedance map obtained through an EIT scan with one or more reference, or control, values e.g. known impedance values for different types of materials and structures or the impedance value of the material being imaged that was taken at an earlier point in time, discrete regions in the resulting image can be identified as being a particular type of material (i.e., malignant tumors, muscle, fat, etc.), as having a particular pH that is distinct from that of the rest of the material, etc. EIT is well developed in the art; see, for example, U.S. Pat. No. 5,919,142, the full disclosure of which is incorporated herein by reference.

As a second example, electrical impedance may be imaged using magnetic electrical impedance tomography (MEIT). MEIT is a modification of EIT that incorporates aspects of magnetic resonance current density imaging (MRCDI) in order to obtain the benefits of both procedures. In MEIT, images are generated by placing a series of electrodes around the material to be imaged, e.g. biological tissue, for the application of current. Note that in contrast to traditional EIT, the electrodes are not contacted with the material, i.e. they are not galvanically coupled with the material. The material, e.g. the patient or object, is placed in a strong magnetic field, and a magnetic resonance imaging sequence is applied which is synchronized with the application of current through the electrodes. The electric potentials of the surface of the object or patient are measured simultaneously with the magnetic resonance imaging sequence, as in EIT. Then, the magnetic resonance imaging signal containing information about the current and the measured potential are processed to calculate the impedance of the material. MEIT is well known in the art, and is described in greater detail in, for example, U.S. Pat. No. 6,397,095, the full disclosure of which is incorporated herein by reference.

According to certain embodiments, the electrolysis process (e.g. the onset of electrolysis, the extent or progression of electrolysis, the cessation of electrolysis, etc.) is detected by measuring and imaging pH changes in a tissue of the subject undergoing electrolysis by magnetic resonance imaging (MRI). In certain aspects, such methods include imaging pH changes in a tissue of the subject undergoing electrolysis by magnetic resonance imaging, and monitoring the electrolysis based on one or more magnetic resonance images of the pH changes in the tissue.

MRI uses magnetic fields and radio waves to produce images of tissues (e.g., images of thin slices of tissues, or “tomographic images”). Normally, protons within tissues spin to produce tiny magnetic fields that are randomly aligned. When surrounded by the strong magnetic field of an MRI device, the magnetic axes align along that field. A radiofrequency pulse is then applied, causing the axes of all protons to momentarily align against the field in a high-energy state. After the pulse, some protons relax and resume their baseline alignment within the magnetic field of the MRI device. The magnitude and rate of energy release that occurs as the protons resume this alignment (T1 relaxation) and as they wobble (precess) during the process (T2 relaxation) are recorded as spatially localized signal intensities by a coil (antenna). Computer algorithms analyze these signals and produce anatomic images.

The relative signal intensity (brightness) of tissues in an MRI image is determined by factors such as the radiofrequency pulse and gradient waveforms used to obtain the image, intrinsic T1 and T2 tissue characteristics, and tissue proton density.

By controlling the radiofrequency pulse and gradient waveforms, computer programs produce specific pulse sequences that determine how an image is obtained (weighted) and how various tissues appear. Images can be T1 (spin-lattice)-weighted, T2 (spin-spin)-weighted, or proton density-weighted. For example, fat appears bright (high signal intensity) on T1-weighted images and relatively dark (low signal intensity) on T2-weighted images; water and fluids appear relatively dark on T1-weighted images and bright on T2-weighted images. T1-weighted images optimally show normal soft-tissue anatomy and fat (e.g., to confirm a fat-containing mass). T2-weighted images optimally show fluid and abnormalities (e.g., tumors, inflammation, trauma). In practice, T1- and T2-weighted images provide complementary information, so both may be employed for characterizing abnormalities.

Details regarding MRI and diagnostic and therapeutic uses thereof are found, e.g., in Westbrook & Roth (2011) MRI in Practice (John Wiley & Sons) (ISBN-10: 1444337432; ISBN-13: 978-1444337433).

In certain aspects, imaging pH changes in a tissue of the subject undergoing electrolysis by magnetic resonance imaging includes imaging pH fronts in the tissue undergoing electrolysis. As demonstrated in Example 2 below, the present inventors have discovered that electrolysis may be imaged/monitored by detecting pH fronts in a tissue undergoing electrolysis. According to certain embodiments, when the imaging technique employed is magnetic resonance imaging, the magnetic resonance images are produced using a sequence selected from: a T1 weighted sequence, a T2 weighted sequence, proton density (PD)-weighted sequence, and combinations thereof. For example, a T1-weighted sequence, a T2-weighted sequence, or a T1- and a T2-weighted sequence may be employed.

In imaging a material by electrical impedance or by MRI, the electrical impedance or MRI measurements may be reconstructed into an image, or map, of the electrical impedance or magnetic resonance of the material and hence of the various regions therein. Towards this end, an image reconstruction algorithm may be employed. For example, when the imaging technique is electrical impedance-based imaging, an image reconstruction algorithm may be used to determine the impedance distribution within a region of interest given a set of current-induced voltage measurements taken at the region's surface (either internal or external). In practicing the subject methods, any convenient reconstruction algorithm may be applied to determine the impedance distribution.

For example, the Newton-Raphson method may be employed. In the Newton Raphson method, a region of interest within the body is identified and geometrically defined. A pattern of electrode placements suitable to this region is then determined, and the absolute electrode positions are measured. Accompanying this electrode arrangement is the data collection algorithm which defines the ordering of the current source/sink and voltage measurement electrode pairs during an image scan. Decisions involving the electrode geometry and data collection algorithm are based upon the imaging region geometry and the specific application, and will ultimately determine the overall attainable image quality.

These pre-procedure definitions are then used to create a mathematical model representing the real imaging region of interest. The model is designed to reflect all relevant bio-electrical physical behavior expected of the real imaging region. That is to say, if the exact impedance distribution of the real region were known, it could be entered into the model and be expected to produce the same voltage measurements as the real system given identical electrode placement and data collection algorithms. This model may then be used as a testing tool for possible impedance distribution candidates by comparing the measured voltages from the real and model regions. The smaller the overall difference in voltage measurements between real and modeled systems, the more closely the modeled impedance distribution represents the real distribution.

Reconstructing an image then become an iterative process involving an initial distribution guess, a testing of that guess via comparison of modeled and real voltage measurements, and a refining of the initial guess based on the comparison results. This process is repeated until the real and modeled measurements are suitably close.

One major component of the Newton-Raphson technique is the modeling method chosen. In practicing the subject methods, any convenient modeling method that finds use in the Newton-Raphson technique may be employed. For example, a finite element approach may be employed, referred to hereafter as an impedance mapping technique. Briefly, this approach approximates a bioelectrical continuum as a set of connected electrically homogeneous elements with enforced boundary continuity. Each element represents an impedance “pixel”. The more elements, the better the image resolution. As a second example, a front tracking technique may be employed. In the front tracking technique, the region of interest is broken down into a number of electrically homogeneous zones defined by a finite number of simply connected boundary segments. The placement of the segment endpoints then define the shape of each zone, with more segments allowing a finer shape resolution. The mathematical method of solution for this model description is known as the boundary element method.

A second major component of the Newton-Raphson technique is the guess refining algorithm. The two things that characterize the type of guess refining algorithm used in the Newton-Raphson method are the parameters which are being refined, and the method of that refinement. Impedance mapping techniques adjust the impedance of each element, whereas the front tracking method adjusts the location of boundary segment endpoints, and therefore the shape of the electrically homogeneous zones. The method of refinement in each case is based on a differential matrix, or Jacobian calculation. This matrix represents the unit change in each measured voltage given a unit change in each element impedance (impedance mapping) or segment end position (front tracking).

One of the major advantages of front tracking over impedance mapping techniques is a drastic decrease in the necessary number of electrodes needed to produce comparable images. Inverse problems of this type are mathematically constrained in that they require at least as many independent voltage measurements as there are adjusting parameters (i.e. elemental impedances or segment end positions). Many imaging applications, such as localized cancers, have fairly simple geometries which can be described well by a small number of shape segments using front tracking. In contrast, impedance mapping would require a comparatively large number of elements, and therefore electrodes, to achieve similar morphological distinction. Front tracking also naturally enforces the expected step changes in impedance across tumor or organ boundaries. Impedance mapping algorithms tend to smooth these boundaries, degrading important morphology features.

One challenging aspect of the front tracking method not present in impedance mapping is the need to “seed” electrically homogeneous zones. That is, before the front tracking algorithm can begin refining a given shape, it needs to know where, how many, and how big the initial zone guesses should be.

A solution to this problem is achieved by combining aspects of the two reconstruction algorithms. A typical sequence demonstrating this would begin by using impedance mapping to roughly identify probable homogeneous zones within the region of interest. These areas would be seeded and the front tracking algorithm would take over in further refinement of each zone's shape until the overall difference between modeled and physical surface voltages was acceptable. Thus, by exploiting the specific strengths of each algorithm, a technique more effective than either the front tracking or impedance mapping technique alone is realized. This combined technique is hereinafter referred to as a hybrid technique.

As demonstrated in the working examples herein, electrical impedance measurements may be used as a proxy, or surrogate, reading of localized changes in pH in a region of a material. Without wishing to be bound by theory, it is believed that this is because changes in pH produce changes in the conductivity of the material. Moreover, it is believed that electrolysis causes a localized change in the pH, electrical impedance can be used to detect and monitor electrolysis in a material.

Imaging technologies that rely on measurements of electrical impedance, e.g. EIT and MEIT, make it possible to produce images of inaccessible regions within a target material based on the spatial variation of the electrical properties of the target material. As such, upon the discovery that electrical impedance measurements may be used to monitor electrolysis within a tissue, such imaging technologies became available as tools for imaging medical manipulations that include tissue electrolysis.

As used herein, tissue electrolysis refers to the delivery of a current between an anode and cathode in a tissue. As used herein, the term “tissue” refers to a plurality of cells. The cells may be of the same or of a number of different types. These cells are preferably organized to carry out a specific function. Tissue includes tissue present within a living organism as well as removed tissue and may refer to in vivo or in vitro situations. Further, the tissue may be from any organism including plants and animals or a tissue developed using genetic engineering and thus be from an artificial source. In one embodiment the tissue is a plurality of cells present within a distinct area of a human. Non-limiting examples of tissues of the subject methods include: brain tissue, lung tissue, heart tissue, muscle tissue, skin tissue, kidney tissue, cornea tissue, liver tissue, abdomen tissue, head tissue, leg tissue, arm tissue, pelvis tissue, chest tissue, prostate tissue, breast tissue, esophagus tissue, GI tract tissue and trunk tissue.

There are a number of applications of electrolysis known in the art. For example, tissue electrolysis may be employed to produce focal, i.e. localized, necrosis, e.g. for the purposes of ablating tissue, e.g. tumor tissue. Electrolytic ablation provides the advantage of safety even when conducted close to major vessels. In some instances, electrolytic ablation may be coupled with radiofrequency in a process referred to as Bimodal electric tissue ablation (BETA), so as to produce larger ablation zones compared to EA or radiofrequency alone while reducing the time required for ablation. Tissue electrolysis for the purposes of tissue ablation is described in e.g. U.S. Pat. No. 7,875,025 and Granvante et al. “Experimental application of electrolysis in the treatment of liver and pancreatic tumours: principles, preclinical and clinical observations and future perspectives.” Surg. Oncol. 2011 June; 20(2):106-20.

As another example, electrolysis may be used in the treatment of ischemic diseases, e.g. ischemic diseases of the eye, for example to treat diabetic retinopathy and ischemia of the retinal and choroidal tissues. As described in, e.g., U.S. Pat. No. 8,655,452, the treatment is based on selective and fractional electrolysis of the vitreous humor to produce oxygen and optionally active chlorine while simultaneously controlling pH. Oxygen or active chlorine can suppress or reverse the onset of diabetic retinopathy, other retinovascular diseases, and choroidal neovascularization.

As another example, electrolysis may be used to promote tissue repair. Without wishing to be bound by theory, it is believed that in this application of tissue electrolysis, e.g. intratissue percutaneous electrolysis (Electrolysis Percutaneous Intratissue (EPI)), the electrolysis promotes inflammation that promotes phagocytosis and repair of affected tissue. See, for example, Abat et al. “Clinical results after ultrasound-guided intratissue percutaneous electrolysis (EPI®) and eccentric exercise in the treatment of patellar tendinopathy.” Knee Surg Sports Traumatol Arthrosc. 2014 Jan. 30 and Gensler W. “Electrochemical healing similarities between animals and plants.” Biophys J. 1979 September; 27(3):461-6.

In some instances, the one or more electrical impedance images or the one or more magnetic resonance images are used to monitor the electrolysis. For example, in some embodiments, an EIT image, MEIT image, or MRI image is generated and the image is used to extrapolate the amount of tissue ablated by an electrolysis process.

In some instances, the one or more electrical impedance images or the one or more magnetic resonance images are used to optimize the electrolysis. For example, a map of electrical impedances essentially allows the user to visualize when electrolysis is beginning. When electrolysis begins the user can stabilize the amount of current being applied and thereby avoid applying unsafe amounts of current. The electrical impedance and magnetic resonance imaging technologies make it possible for the region of tissue undergoing electrolysis to be visualized based on changes in equivalent electrical impedance of the cells or pH changes within tissue being monitored.

In certain aspects, algorithms such bulk measurements or classifiers using the same or similar principles are employed to monitor electrolysis, obviating any need to produce an image in order to monitor electrolysis.

### Devices and Systems

Also provided are devices and systems for practicing one or more of the above-described methods. The subject devices and systems thereof may vary greatly. Devices and systems of interest include those mentioned above with respect to the methods of EIT, MEIT, and MRI. According to certain embodiments, a system for performing tissue electrolysis in an individual is provided. The system includes an electrolytic device and an electrical impedance measuring device. The measuring device may be an electrical impedance imaging device, or a measurement device that takes bulk measurements of electrical properties and monitors their changes with electrolysis, or monitors bulk changes in magnetic resonance readings and their changes with electrolysis.

When the imaging technique employed is electrical impedance-based imaging, the subject devices and systems may include one or more of an electrical impedance imaging device, an electrolytic device, a power source, e.g. as described herein or as known in the art. The terms “electrical impedance imaging device” as used herein refers to any device as described herein or as known in the art that finds use in imaging electrical impedance in a material, for example, an electrical impedance tomography (EIT) device, a magnetic resonance electrical impedance tomography (MEIT) device”, etc. In some embodiments, the subject imaging device comprises 2 or more, 3 or more, 4 or more, 5 or more, 6 or more, 7 or more, 8 or more, 9 or more, 10 or more, 50 or more, 100 or more, 200 or more, 400 or more electrodes, for example, 2, 4, 8, 16, 32, 64, 128, 256, or 512 electrodes and typically not more than about 600 electrodes. By “electrode” is intended to mean any electrically conductive material, preferably a metal, most preferably a non-corrosive metal that is used to establish the flow of electrical current or voltage from that electrode to another electrode, e.g. in electrical impedance tomography or magnetic resonance electrical impedance tomography. Electrodes serve as an electrically conductive means for transmitting electrical current that can be referred to in any manner, e.g. current or voltage. Electrodes are made of a variety of different electrically conductive materials and may be alloys or pure metals such as copper, gold, platinum, steel, silver, silver chloride, and alloys thereof. Further, the electrode may be comprised of a non-metal that is electrically conductive such as a silicon-based material used in connection with microcircuits. Typical electrodes used in tissue electrolysis are preferably rod-shaped, flat plate-shaped or hollow needle-shaped structures. Electrodes may be used to deliver electrical current continuously or to deliver pulses. The electrodes may be very application-specific and be comprised of parallel stainless steel plates, implanted wires, needle pairs and needle arrays. Examples of arrangements of electrodes are well known in the art; see, for example, U.S. Pat. No. 6,501,984. In some instance, the electrode may be an electrically conductive solid ring electrode; see, for example, U.S. Pat. No. 6,940,286, which describes methods and an apparatus for obtaining a representation of the distribution of electrical impedance within a multiphase flow with an electrically continuous or discontinuous principle flow contained within an electrically conductive solid ring electrode; the full disclosures of which is incorporated herein by reference. Those skilled in the art will design specific electrodes, coils, or antennae that are particularly useful in connection with the desired results of obtaining electrolysis in accordance with the present invention.

When the imaging technique employed is magnetic resonance imaging (MRI), the subject devices and systems may include one or more of a magnetic resonance imaging device, an electrolytic device, a power source, e.g. as described herein or as known in the art. Any suitable device for producing magnetic resonance images may be employed. For example, the magnetic resonance imaging device may be a device marketed and sold by GE Healthcare (e.g., a Discovery, Optima, Brivo, or Signa MRI device), Hitachi Medical Systems (e.g., an Oasis or Echelon MRI device), Toshiba Medical Systems (e.g., a Vantage MRI device), Siemens Healthcare (e.g., a Magnetom MRI device), and Philips Healthcare (e.g., an Ingenia, Achieva, Multiva, or Sonalleve MRI device).

The terms “electrolysis device” or “electrolytic device” as used herein refer to any device as described herein or as known in the art that finds use in the electrolysis of tissue. The device preferably includes a first electrode and a second electrode wherein the first and second electrodes are connected to a source of electricity in a manner so as to provide the electrodes with positive and negative charges respectively. In some instances, the electrode providing the current to the tissue is the cathode. In other instances, the electrode providing the current to the tissue is the anode. Non-limiting examples of electrolytic devices that find use in the subject methods, devices and systems include those disclosed in U.S. Pat. No. 7,875,025, U.S. Pat. No. 8,655,452, and U.S. Pat. No. 7,819,864.

The electrolytic device may also include a means for hindering the flow of electricity between the two electrodes except through one or more specific openings. For example, the means for hindering flow can be non-conductive material which has one or more openings therein wherein the openings are designed so as to specifically hold a biological cell or group of biological cells. Thereby the electrical current must flow through the opening and through the cells to the other electrode. The device also preferably includes a means for measuring the flow of electrical current between the electrodes. The means for measuring can include a volt meter, amp meter or any device known to those skilled in the art which is capable of measuring the flow of electrical current in any manner. Further, in some embodiments, the device includes a means for adjusting the amount of electrical current flow between the electrodes. Thereby the voltage, current or other desired parameter of electrical current flow can be specifically adjusted based on the measured flow so as to obtain optimum electrolysis of the cell or cells positioned between the electrodes.

The terms “power source”, “source of electricity” and the like, are used interchangeably herein to describe any means for providing electrical power, current or voltage thereby creating a flow of electrical current between the electrodes. The device preferably is capable of providing for a controlled mode and amplitude and may provide constant DC current or AC current, provide pulse voltage or continuous voltage. In some instances, the devices are capable of exponentially decaying voltage, ramp voltage, ramped current, or any other combination. For example, a power supply may be used in combination with a chip of the type used in connection with microprocessors and provide for high-speed power amplification in connection with a conventional wall circuit providing alternating voltage. The pulse shape may be generated by a microprocessor device such as a Toshiba laptop running on a LabView program with output fed into a power amplifier. A wide range of different commercially-available power supplies can provide the desired function.

For example, the power supply may be a component of an electrolysis device. In such instances, the electrical stimulation delivered for electrolysis is usually quoted in terms of the current supplied to a region, with a magnitude of current typically within a range of about 1 μAmp/cm² to 100 mAmp/cm² e.g. 100 μAmp/cm² to 5 mAmp/cm², for example, 100 μAmp/cm² to 500 μAmp/cm², 500 μAmp to 1 mAmp, 1 mAmp-5 mAmp; where the surface area is the surface area of the electrode. However, the range is amplification-specific and can be extended outside the range for any desired application. The current may be direct current or alternating current; more usually, the current will be direct current. Typically, the current will be applied continuously, e.g. for 1 second or more, 10 seconds or more, 20 seconds or more, 30 seconds or more, 40 seconds or more, 50 seconds or more, 1 minute or more, 2 minutes or more, 4 minutes or more, 5 minutes or more, 10 minutes or more, or 15 minutes or more. In certain aspects, the current is applied for hours (e.g., 1 or more hours, 2 or more hours, 3 or more hours, 4 or more hours, 5 or more hours, 12 or more hours, etc.) or days (e.g., 1 or more days, 2 or more days, 3 or more days, 4 or more days, 5 or more days, etc.). This is in contrast to electroporation, in which microsecond pulses of prescribed electric field strength, e.g. 100-10,000 volts/cm, are delivered; see, for example, U.S. Pat. No. 6,725,087 to Rubinsky. As will be appreciated by the ordinarily skilled artisan, other ranges of currents and lengths of stimulation may be utilized to promote electrolysis, depending on the desired results.

As a second example, the power supply may be a component of the electrical impedance imaging or magnetic resonance imaging device. For example, the stimulation signal, when applied to the tissue, produces a potential field in the volume which is then detected by the measurement electrodes. Electrical stimulation for the purposes of electrical impedance imaging is typically a subsensory stimulation, e.g. about 50 μAmps-500 μAmps, e.g. of alternating current delivered over milliseconds, e.g. about 1-10 milliseconds. Other combinations of current and time may be applied as well, depending on the desired results. See, for example, U.S. Pat. No. 5,381,333, U.S. Pat. No. 5,919,142, U.S. Pat. No. 6,236,886, U.S. Pat. No. 6,387,671, and U.S. Pat. No. 6,397,095, the disclosures of which are incorporated herein by reference.

In addition to the above components, the devices and systems may further include instructions for practicing the methods of the present disclosure. These instructions may be present with the subject devices and systems in a variety of forms, one or more of which may be present in the subject device or system. One form in which these instructions may be present is as printed information on a suitable medium or substrate, e.g., a piece or pieces of paper on which the information is printed, in the packaging of the device or systems, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the subject device or system.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1—Monitoring Electrolysis Using Electrical Impedance Tomography (EIT)

Tissue ablation with minimally invasive surgery is important for treatment of many diseases and has an increasing role in treatment of solid neoplasms. A variety of biophysical and biochemical processes are used for this purpose. They include thermal ablation with heating, cooling or freezing, electroporation, injection of chemical agents, photodynamic effects, sonoporation effects and many others.

Electrolysis, the passage of a low amperage direct ionic current through the tissue, between two electrodes, is a biochemical/biophysical process that has been considered for tissue ablation since the 19th century [1]. Electrolysis affects the ionic species in tissue, which change into compounds that can ablate cells. Electrolysis is currently limited by the lack of an effective means to monitor the extent of tissue ablation deep in the body.

The work of Nordenstrom and colleagues [2, 3] is among the early modern work on electrolysis. Important recent work was published on understanding the effects of electrolysis on tissue through histology, mathematical modeling of involved electrochemical processes, and clinical work, e.g. [4-14] and [15-18]. It has been shown that the electrolysis-induced pH changes can be used to reliably monitor the extent of tissue ablation [19]. These findings have led to several basic studies on quantifying the process of electrolysis through the use of transparent gels with pH dyes [11, 20, 21].

Electrical impedance tomography (EIT) is used in a variety of scientific fields, from geology, to semiconductor characterization, to medical imaging. EIT produces an image of the electrical properties of the examined media. In a typical EIT application, electrodes are placed around the volume of interest, and small, sinusoidal currents are injected into the tissue, while voltages are measured on its boundary. Using the finite element method, the complex impedance of the analyzed domain is modeled, and a solution for the most likely impedance configuration that fits the measurements is obtained [26-28]. EIT-based techniques have been applied to monitor minimally invasive surgery procedures such as cryosurgery [29], tissue viability [30, 31] and electroporation [32],[33].

The present study reports the first use of electrical impedance tomography (EIT), an imaging technique that produces a map of electrical properties, as a means to image dynamic changes in local pH level of a biological sample during an electrolytic process. This study uses a pH dye stained agar-gel based phantom as a model for a living tissue, from an electrochemical standpoint. To investigate the concept, EIT reconstructed images were compared to optical images acquired using pH-sensitive dyes embedded in the agar phantom. In addition to validating the EIT-based approach using pH-sensitive dyes, we demonstrate a biological application of our EIT work by comparing a spatial map of bacterial viability exposed to electrolysis with the EIT image of the phantom during electrolytic treatment. The experimental findings demonstrate the feasibility of using EIT, and more broadly, electrical impedance imaging, as a means to image dynamic changes in local pH level of a biological sample during an electrolytic process, and hence, for example, to monitor electrolytic surgery in real time. The study has relevance to real time control of minimally-invasive surgery with electrolytic ablation.

### Materials and Methods

A. Tissue Model

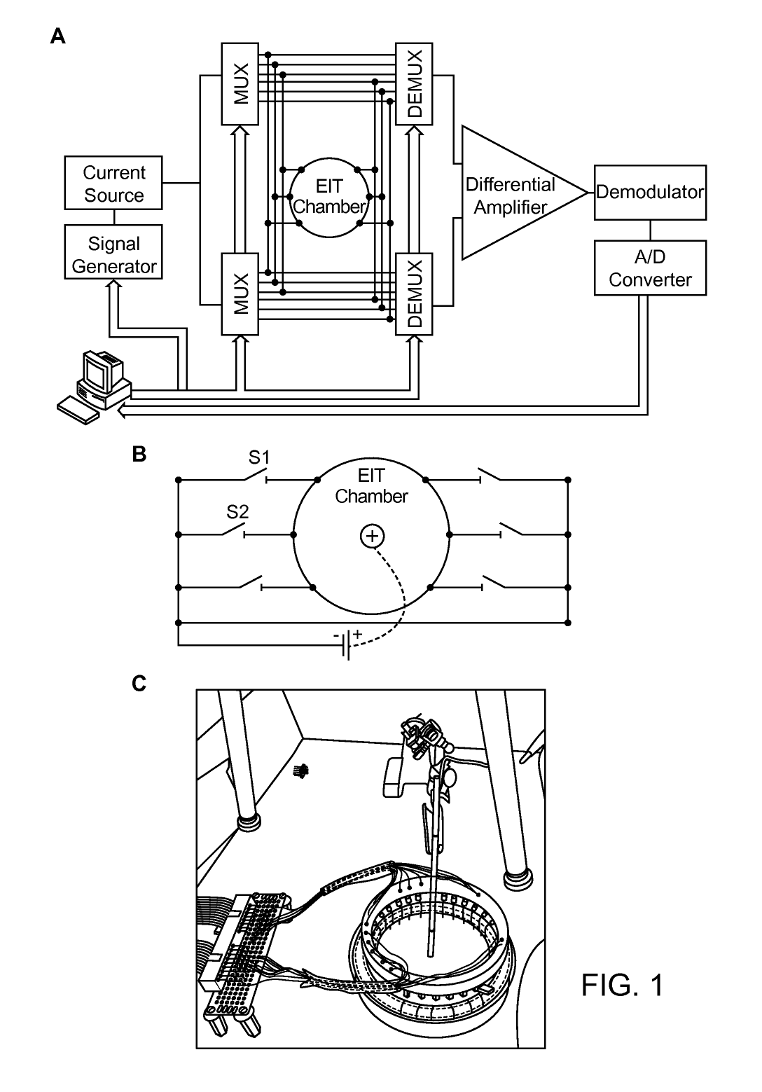

The tissue model consists of a physiological saline based agar gel phantom with electrical conductivity designed to simulate that of a tissue. To construct the phantom, 0.5% Bacto-Agar (Fisher Scientific) was mixed with 0.9 g/I Sodium Chloride (Fisher Scientific) in distilled water. The solution was then brought to a boil and poured into the Petri dishes. The conductivity of the agar phantom was measured to be approximately 0.14 S/m which is close to the range of hepatic tumor conductivity [34]. During the experiments, the EIT electrode holder was placed in the Petri dish with the electrodes galvanically coupled to the gel phantom (FIG. 1).

B. Experimental Model

To test the feasibility of EIT as a means to monitor the onset and extent of electrolysis in tissue, the following experiment was devised: 1) a reference EIT image of the tissue phantom is taken; 2) electrolytic stimulation is applied; and 3) another EIT reference of the tissue phantom is taken. We leverage the differential nature of EIT images to represent the changes in conductivity, which are used as surrogates to regions of altered pH level. As a control study, we use pH sensitive dyes in order to estimate the boundary of the region where the pH has changed due to electrolysis. We use a digital camera (Casio Exilim EX-ZR100) to acquire optical images of the experimental chamber and correlate these images with the EIT reconstruction images. The results of several representative studies are presented in the following section: in each study we have repeated experimental steps 2) and 3), above, multiple times, in order to observe the evolution of the pH front over time.

C. Bacterial Model

Lyophilized E. coli of HB101 strain (BioRad) were grown in LB broth overnight and plated on LB broth based agar gel filled petri dishes. The LB broth for the overnight growth consisted of 1% BactoTryptone (BD), 0.5% Yeast Extract (BD), 1% NaCl (Sigma Aldrich) and 1.5% Agarose (Sigma Aldrich). For pouring the plates, we held the sodium salt from the broth, in order to control the conductivity of the resulting gel. 6 mm glass beads (Sigma Aldrich) were used for plating to ensure uniform coverage. After plating, the beads were removed and the plates were incubated for 15 minutes at 37° C. The conductivity of the gel was measured around 0.2 S/m. At the experimental stage, the petri dish was separated from its lid and the EIT electrode array was lowered into the gel. On top of the EIT chamber, a 2 electrode holder with auxiliary electrodes was introduced into the gel. For the bacteria-focused experiments, only the auxiliary electrodes were used for stimulation, as opposed to the pH-sensitive dye experiments where we have also used the EIT electrodes for electrolytic stimulation. The stimulation sequence was applied using a specified current and time parameters, with EIT snapshots being taken in the process as a monitoring step. After the stimulation, the petri dishes were covered and incubated for 24 hours. To evaluate viability we have visually inspected the petri dishes for areas where bacterial growth was inhibited.

D. EIT Instrumentation

An EIT data acquisition system consists of a collection of electrodes, which are used to inject known sinusoidal AC current into the observed sample. Due to the sample's conductivity, a potential develops on the sample. This potential is measured on the boundary using the electrodes not used for current injection. A schematic of a typical EIT system is presented in FIG. 1.a. In this work, we have used the EIT system described in [34], with N=32 electrode surrounded circular chamber. We used an adjacent stimulation scheme [35], leading to each data set containing (N·(N−3))/2=464 independent measurements. After the data has been acquired, the data processing module of an EIT system attempts to reconstruct a conductivity map of the domain of interest from a set of known injected current measured resulting voltages, typically at the boundary of the geometric domain. In a typical EIT reconstruction algorithm, a map of impedance is guessed and the voltages resulting from injected currents calculated by solving Laplace equation in the domain. These voltages are compared to the measured voltage and the difference is then used as feedback for an iterative scheme. The guessed map of impedance is the updated, until the calculated and measured voltages agree within a certain tolerance. Here, a modified Newton-Raphson (NR) method was used for reconstructing the image from the input data due to its excellent convergence properties [36]. This method attempts to iteratively minimize a cost function representing the overall voltage measurement discrepancy between the input (measured) voltages and the reconstruction algorithm's internal model. The Jacobian needed for the NR method was calculated using a sensitivity matrix approach [37]. Total Variation regularization was used to overcome the ill conditioning of the Jacobian matrix [38].

E. Experimental Setup

The system is composed of 32 stainless steel electrodes mounted on a holder (Diameter=75 mm) lowered into a circular Petri dish (diameter=85 mm) chamber (FIG. 1.c). The chamber contains the pH dye infused agar gel phantom which is imaged using EIT and optical digital camera. All the EIT stimulation currents had amplitude of 350 pA.

### Results

### A. Anode Centered Experiment

In this experiment, a thin, stainless steel rod (diameter 0.6 mm) was placed in the center of the agar gel filled chamber. The central rod was connected using a copper wire to the positive terminal of the power supply and acted as the anode during this part of the experiment. For the cathode, all the 32 electrodes of the EIT were connected to each other by closing the switches S1 . . . S32 presented schematically in the diagram on FIG. 1.b. The negative power supply terminal was then connected to the unified EIT electrodes. The EIT electrodes acted a distributed cathode in this case. As a control study we have employed two pH sensitive dyes: 1% phenolphthalein (Sigma-Aldrich) which turns pink/purple above pH 8.8 and acts as a basic indicator, and 2.4% pH indicator (Fresh water test-kit, API) which turns yellow at pH 6.0. Both pH indicators were added to the agar gel phantom before its solidification.

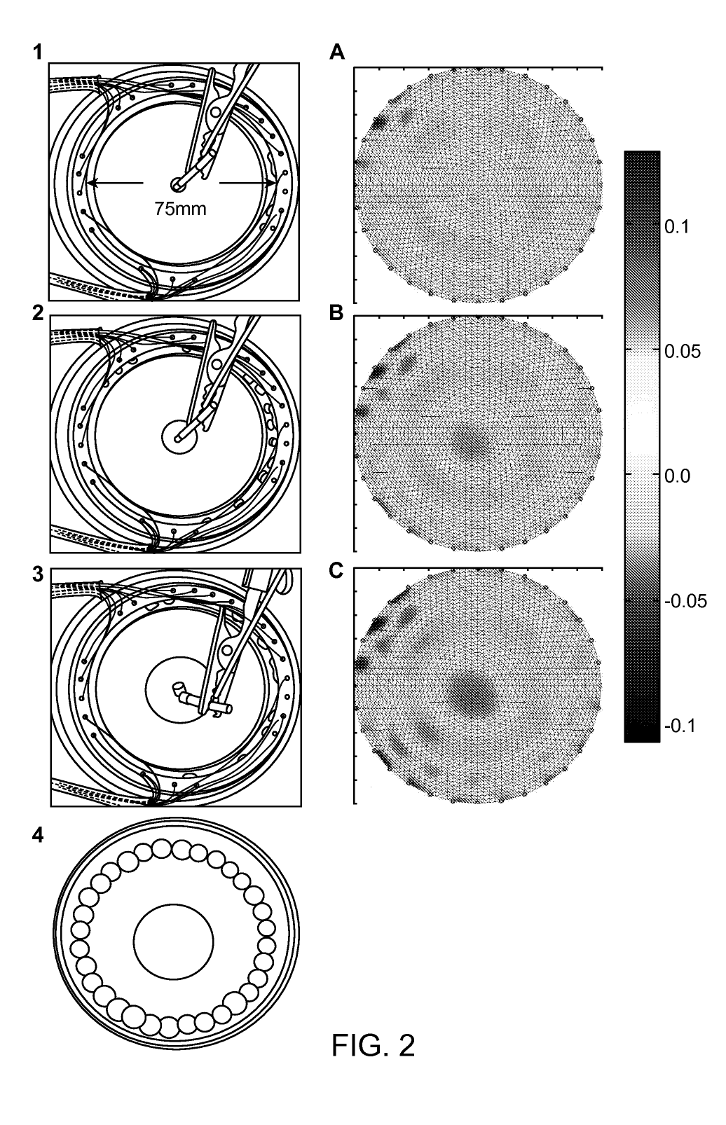

A photo of the experimental chamber is presented in FIG. 1c. The protocol of our experiment involved taking a control set of images: EIT and optical, before every electro-stimulation step. The electro-stimulation included a sequence of direct current injections at 1 mA of the following durations: [1 min, 1 min, 1 min, 1 min, 1 min, 5 min, 10 min]. These parameters are typical to tissue ablation electrolytic processes, at the lower range of the parameters [3, 40]. FIG. 2 summarizes the results of our experiment by showing a sequence of image pairs: each EIT image is accompanied by its matching optical image which we used as a validation method.

The current was delivered at 1 mA, and the delivered charge dosage was 1.14c, which falls within a range of a typical electro-chemo therapy stimulation charge dosage [3, 40]. FIGS. 2.a-2.c show the EIT images at selected time points whereas FIGS. 2.1-2.3 show the corresponding optical images. FIG. 2.4 shows the final result of the gel model after the EIT electrodes have been removed. It can be seen that the EIT images of the pH from near the central electrodes are in good correspondence with the pH indicator dye: the central spot around the anode grows over time in both the optical and the EIT images. The data shows a good qualitative correspondence between the EIT reconstructed images and their optical counterparts. We have chosen to include representative images corresponding to times t=1 minutes, t=3 minutes and t=19 minutes. The contrast of the image in FIG. 2.4 was increased to show the altered pH indicator at the perimeter, close to the distributed anode.

The color bar presented to the right of the figure facilitates interpretation of the EIT results: the EIT images are taken in differential mode which means that the images show differences relative to a reference image taken before any electrolytic stimulation was applied. Warmer colors correspond to increased conductivity while colder colors correspond to decreased conductivity in the sample.

B. Cathode Centered Experiment

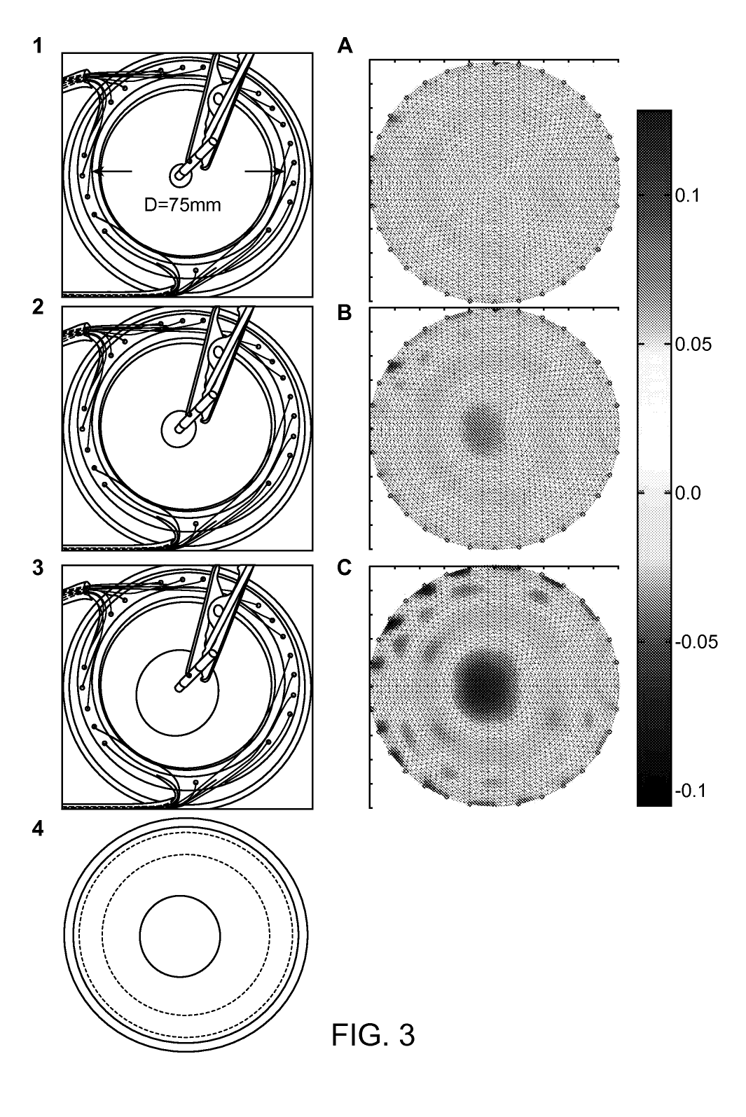

In this part of the experiment, we have reversed the roles of the anode and the cathode. The same pH indicators were used as before: 1% phenolphthalein which turns pink/purple above pH 8.8 and acts as a basic indicator, and 2.4% pH indicator (Fresh water test-kit, API) which turns yellow at pH 6.0. As in the previous section, both pH indicators were added to the agar gel phantom before its solidification. The protocol of this experiment involved taking another control set of images: EIT and optical, before every electro-stimulation step. The electro-stimulation included a sequence of direct current injections at 1 mA of the following durations: [1 min, 1 min, 1 min, 1 min, 1 min, 1 min, 5 min, 5 min, 5 min, 10 min, 10 min]. FIG. 3 presents the results of the experiment by showing a sequence of image pairs: each EIT image is accompanied by its matching optical image which we used as a validation method. The overall charge dosage was charge dosage was 2.16 C. While this dosage falls within a range of a typical electro-chemo therapy procedure, it is a larger charge dosage compared to the anode centered experiment. We have administered more charge in the cathode-centric experiment because the altered pH front indicated by the pH-sensitive dye (phenolphthalein) was growing slower in the cathode-centered case. A possible explanation to this difference is the relative size of the H⁺ and the OH⁻ ions, and this discrepancy is discussed in more detail in a later section (Bacterial Sterilization Model). FIGS. 3.a-3.c show the EIT images at selected time points whereas FIGS. 3.1-3.3 show the corresponding optical images. FIG. 3.4 shows the final result of the gel model after the EIT electrodes have been removed. It can be seen that the EIT images are in good correspondence with the pH indicator dye: the central spot around the cathode grows over time in both the optical and the EIT images. Moreover, while it is too subtle to see in the optical images, the EIT imaging clearly shows a circular feature at the periphery of the EIT chamber. This peripheral region with reduced pH level can be clearly distinguished in FIG. 3.4 by its distinguished yellowish color. It can only be seen after the EIT electrodes have been removed. The data shows a good qualitative correspondence between the EIT reconstructed images and their optical counterparts. Representative images corresponding to times t=2 minutes, t=6 minutes and t=36 minutes are included. An accumulation of liquid, presumed to be water can be observed around the cathode in the form of a growing bubble. We have attributed it to the osmotic effects of electrolysis reported by other researchers. FIG. 3.4 shows the optical image after the EIT electrodes have been lifted at the end of the experiment. The contrast of the image in FIG. 3.4 was increased to show the altered pH indicator at the perimeter, close to the distributed anode. Strikingly, EIT was able to detect the changes in pH along the distributed anode much earlier than they could be detected optically without image processing techniques.

C. Two Internal Electrodes Experiment

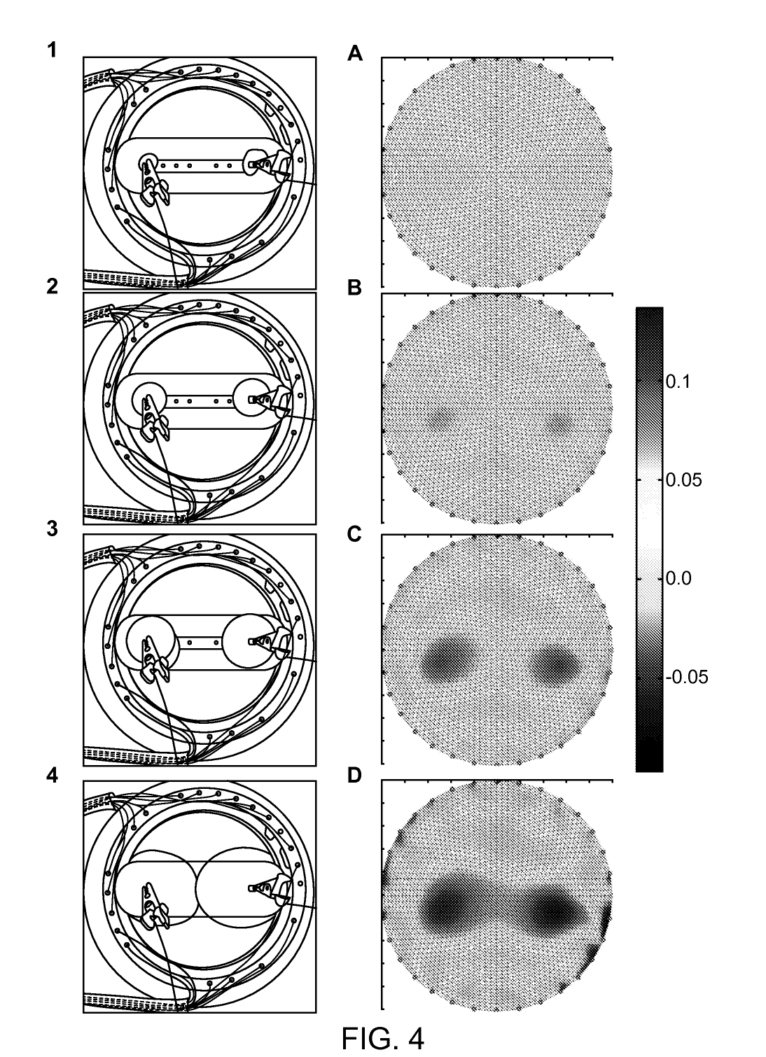

In this part of the experiment, instead of using the EIT electrodes as a distributed electrode, we have utilized two graphite electrodes made of pencil lead (Pentel super HB 0.7 mm). The electrodes, mounted in a horizontal holder were placed perpendicularly to the gel phantom. The electrodes were inserted 5 mm deep into the gel. We have used a 5% pH indicator (RC Hagen wide range). As in the previous experiments, the pH indicator was added to the agar gel phantom before its solidification. The protocol of this experiment involved taking another control set of images: EIT and optical, before every electro-stimulation step. The electro-stimulation included a sequence of direct current injections at 2 mA of the following durations: [1 min, 1 min, 1 min, 1 min, 1 min, 1 min, 1 min, 5 min]. FIG. 4 presents the results of the experiment by showing a sequence of image pairs: each EIT image is accompanied by its matching optical image which we used as a validation method. The calculated current density at each one of the electrodes can be estimated as I/A=2 mA/(2πrd)=21.26 mA/(cm̂2) which falls within a range of a typical electro-chemo therapy stimulation current density. FIGS. 4.a-4.c show the EIT images at selected time points whereas FIGS. 4.1-4.3 show the corresponding optical images. It can be seen that the EIT images are in good correspondence with the pH indicator dye: the central spot around the anode (red) grows over time in both the optical and the EIT images, and the same is observed for the spot around the cathode (blue). We have chosen to include representative images corresponding to times t=1 minutes, t=3 minutes, t=6 minutes and t=12 minutes. It is notable that both the optical and the EIT approaches are able to image the collision of the basic and the acidic fronts (FIGS. 4.d and 4.4).

D. Bacterial Sterilization Model

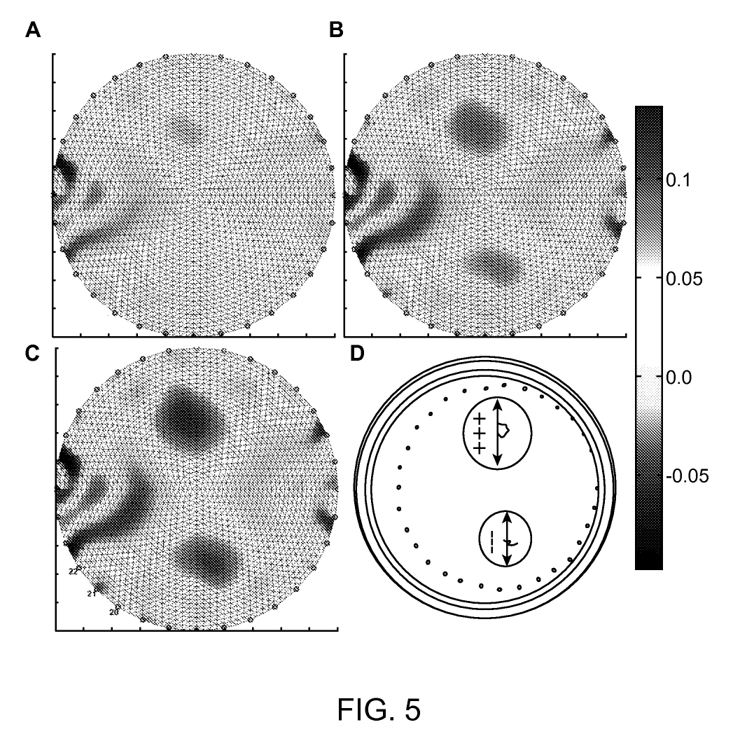

To confirm the efficacy of our method in a biological model, we have used EIT for imaging electrolysis in an agar dish plated with E. Coli bacteria. The liquid bacterial culture was first plated as described in our methods, and then a current of 2 mA was administered using the auxiliary electrodes. The total administered charge dosage was 5.4 C which falls within a range of a typically delivered charge during an electro-chemo therapy stimulation [3, 40]. FIG. 5 shows a comparison between the EIT imaging data and a bacterial viability pattern captured using an optical, digital camera after 24 hour growth period. FIGS. 5.a-5.c indicate two growing regions of increased conductivity, around the auxiliary electrodes through which electrolytic stimulation was applied. We have chosen to include representative images corresponding to times t=15 minutes, t=30 minutes and t=45 minutes. FIG. 5.d shows the optical image of the viability pattern taken 24 hours post-stimulation. It is interesting to note that both the EIT images as well as the optical image exhibit asymmetry with regards to the anodic and the cathodic regions under our experimental conditions. The brighter upper spots in the EIT images, in particular in the one shown in FIG. 5.c indicates that the conductivity of the anodic region has changed to a larger degree than the conductivity of the cathodic region. This discrepancy can be attributed to the relative radii of protons (H+, 0.88 fm) and hydroxide ions (OH−, 110 pm). Due to their relative smaller size, the protons are more mobile hence contributing to a larger extent to the conductivity increase around the anode. The increased mobility causes the bactericidal pH region around the anode to be larger than around the cathode. This is supported by the viability observations presented in FIG. 5.d. To clarify, the circular pattern of dots around the bacterial culture dish corresponds to the EIT electrodes imprinted in the gel when the EIT chamber was lowered.

### Discussion

In summary, we report experimental findings that support the hypothesis that electrolysis induced pH-changes lead to local conductivity changes in a physiological gel tissue model. It is these changes in conductivity that can be captured in real time by EIT. Our results indicate the feasibility of using EIT as a means to monitor dynamic changes in local pH level of a biological sample during an electrolysis process. Our work uses agar-based gel model with conductivity in the range of a biological tissue, and is validated vs. optical images utilizing pH indicator dyes. In addition we demonstrate the relevance of our work in the biological context by correlating bacterial viability data with EIT measurements.
[1] R. Amory, A treatise on electrolysis and its therapeutical and surgical treatment in disease. New York: William Woof &Co., 1886.
[2] B. E. W. Nordenstrom, “Preliminary clinical trials of electrophoretic ionization in the treatment of malignant tumors.,” IRCS Medical Sc., vol. 6, p. 537, 1978.
[3] B. E. Nordenstrom, “Electrochemical treatment of cancer. I: Variable response to anodic and cathodic fields,” American journal of clinical oncology, vol. 12, pp. 530-536, 1989.
[4] E. Nilsson, et al., “Development of a dosage method for electrochemical treatment of tumours: a simplified mathematical model,” Bioelectrochemistry and Bioenergetics, vol. 47, pp. 11-18, November 1998.
[5] E. Nilsson, et al., “Electrochemical treatment of tumours: a simplified mathematical model,” Journal of Electroanalytical Chemistry, vol. 460, pp. 88-99, Jan. 18, 1999.
[6] E. Nilsson, et al., “Electrochemical treatment of tumours,” Bioelectrochemistry, vol. 51, pp. 1-11, February 2000.
[7] H. von Euler, et al., “Electrochemical treatment (EchT) effects in rat mammary and liver tissue. In vivo optimizing of a dose-planning model for EChT of tumours,” Bioelectrochemistry, vol. 54, pp. 117-124, November 2001.
[8] H. von Euler, et al., “Development of a dose-planning method for electrochemical treatment of tumors: A study of mammary tissue in healthy female CD rats,” Electro- and Magnetobiology, vol. 18, pp. 93-+, 1999 1999.
[9] A. E. Bergues Pupo, et al., “Analytical and numerical solutions of the potential and electric field generated by different electrode arrays in a tumor tissue under electrotherapy,” Biomedical Engineering Online, vol. 10, Sep. 24, 2011.
[10] R. Placeres Jimenez, et al., “3D Stationary Electric Current Density in a Spherical Tumor Treated With Low Direct Current: An Analytical Solution,” Bioelectromagnetics, vol. 32, pp. 120-130, February 2011.
[11] P. Turjanski, et al., “pH front tracking in the electrochemical treatment (EChT) of tumors: Experiments and simulations,” Electrochimica Acta, vol. 54, pp. 6199-6206, Nov. 1, 2009.
[12] H. M. Camue Ciria, et al., “Antitumor effects of electrochemical treatment,” Chinese Journal of Cancer Research, vol. 25, pp. 223-234, April 2013.
[13] D.-S. Yoon, et al., “Introduction of electrochemical therapy (EChT) and application of EChT to the breast tumor,” Journal of Breast Cancer, vol. 10, pp. 162-168, June 2007.
[14] R. Czymek, et al., “Electrochemical Treatment: An Investigation of Dose-Response Relationships Using an Isolated Liver Perfusion Model,” Saudi Journal of Gastroenterology, vol. 17, pp. 335-342, September-October 2011.
[15] D. Griffin, et al., “Low-level direct electrical current therapy for hepatic metastases. I. Preclinical studies on normal liver,” British journal of cancer, vol. 72, pp. 31-34, 1995.
[16] D. Griffin, et al., “The effects of low-level direct current therapy on a preclinical mammary carcinoma: tumour regression and systemic biochemical sequelae,” British journal of cancer, vol. 69, pp. 875-878, 1994.
[17] D. Miklav\check{c}i\check{c}, et al., “Tumor treatment by direct electric current-tumor temperature and pH, electrode material and configuration,” Bioelectrochemistry and bioenergetics, vol. 30, pp. 209-220, 1993.
[18] D. Miklav\check{c}i\check{c}, “Tumour treatment by direct electric current: electrode material deposition,” Bioelectrochemistry and Bioenergetics, vol. 35, pp. 93-97, 1994.
[19] J. G. Finch, et al., “Liver electrolysis: pH can reliably monitor the extent of hepatic ablation in pigs,” Clinical Science, vol. 102, pp. 389-395, April 2002.
[20] N. Olaiz, et al., “Tracking protein electrodenaturation fronts in the electrochemical treatment of tumors,” Electrochemistry Communications, vol. 12, pp. 94-97, January 2010.
[21] M. L. A. Ivic, et al., “An electrochemical illustration of the mathematical modelling of chlorine impact and acidification in electrochemical tumour treatment and its application on an agar-agar gel system,” Journal of Electroanalytical Chemistry, vol. 549, pp. 129-135, Jun. 5, 2003.
[22] K. F. Chu and D. E. Dupuy, “Thermal ablation of tumours: biological mechanisms and advances in therapy,” Nature Reviews/Cancer, vol. 14, pp. 199-208, 2014.
[23] P. Turjanski, m et al., “pH front tracking in the electrochemical treatment (EChT) of tumors: Experiments and simulations,” Electrochimica Acta, vol. 54, pp. 6199-6206, 2009.
[24] P. Turjanski, et al., “The role of pH fronts in reversible electroporation,” PloS one, vol. 6, p. e17303, 2011.
[25] F. Maglietti, et al., “The Role of Ph Fronts in Tissue Electroporation Based Treatments,” PloS one, vol. 8, p. e80167, 2013.
[26] J. T. Yorkey and J. G. Webster, “A comparison of impedance tomographic reconstruction algorithms.,” Clin. Phys. Physiol. Meas., vol. 8, pp. 55-62, 1987.
[27] K. Boone, et al., “Imaging with electricity: Report of the European Concerted Action on Impedance Tomography,” Journal of Medical Engineering & Technology, vol. 21, pp. 201-232, November-December 1997.
[28] D. Barber and B. Brown, “Applied potential tomography,” Journal of physics. E. Scientific instruments, vol. 17, pp. 723-733, 1984.
[29] R. V. Davalos, et al., “A feasibility study for electrical impedance tomography as a means to monitor tissue electroporation for molecular medicine,” Ieee Transactions on Biomedical Engineering, vol. 49, pp. 400-403, 2002.
[30] J. F. Edd and B. Rubinsky, “Assessment of the viability of transplant organs with 3D electrical impedance tomography,” 2005 27th Annual International Conference of the IEEE Engineering in Medicine and Biology Society (IEEE Cat. No. 05CH37611C), pp. 4 pp.|CD-ROM, 2006.
[31] R. Davalos and B. Rubinsky, “Electrical impedance tomography of cell viability in tissue with application to cryosurgery,” Transactions of the ASME. Journal of Biomechanical Engineering, vol. 126, pp. 305-9, 2004.
[32] R. V. Davalos, et al., “Electrical impedance tomography for imaging tissue electroporation,” Biomedical Engineering, IEEE Transactions on, vol. 51, pp. 761-767, 2004.
[33] Y. Granot, et al., “In vivo imaging of irreversible electroporation by means of electrical impedance tomography,” Physics in Medicine and Biology, vol. 54, pp. 4927-4943, 2009.
[34] Y. Granot, et al., “In vivo imaging of irreversible electroporation by means of electrical impedance tomography,” Physics in medicine and biology, vol. 54, p. 4927, 2009.
[35] B. Brown, “Electrical impedance tomography (EIT): a review,” Journal of medical engineering & technology, vol. 27, pp. 97-108, 2003.
[36] T. Yorkey and J. Webster, “A comparison of impedance tomographic reconstruction algorithms,” Clinical Physics and Physiological Measurement, vol. 8, p. 55, 1987.
[37] N. G. Gencer, et al., “Electrical impedance tomography: induced-current imaging achieved with a multiple coil system,” Biomedical Engineering, IEEE Transactions on, vol. 43, pp. 139-149, 1996.
[38] D. W. Marquardt, “An algorithm for least-squares estimation of nonlinear parameters,” Journal of the Society for Industrial & Applied Mathematics, vol. 11, pp. 431-441, 1963.

### Example 2—Monitoring Electrolysis Using Magnetic Resonance Imaging (MRI)

### Materials and Methods

Experimental Outline

The experiment was designed to allow for a comparison between different images of pH fronts produced by the electrolysis of a physiological saline solution phantom. The images were generated by various MRI sequences and compared with: a) optical images acquired using pH-sensitive dyes embedded in a physiological saline agar solution phantom treated with electrolysis; and b) a bacterial E. coli model, grown on a phantom and treated by applying the same electrolysis protocol. Each experimental study was carried out separately.

A. Tissue Model

The tissue model consists of a physiological saline based agar gel phantom with electrical conductivity designed to simulate that of a tissue. To construct the phantom, 1% Bacto-Agar (Fisher Scientific) was mixed with 0.9 g/I Sodium Chloride (Fisher Scientific) in distilled water. The solution was then brought to a boil and poured into 85 mm diameter Petri dishes. The same dish dimension was used in all of the studies. The conductivity of the agar phantom was measured to be approximately 0.14 S/m which is close to the range of hepatic tumor conductivity³⁷.

B. Experimental Procedure

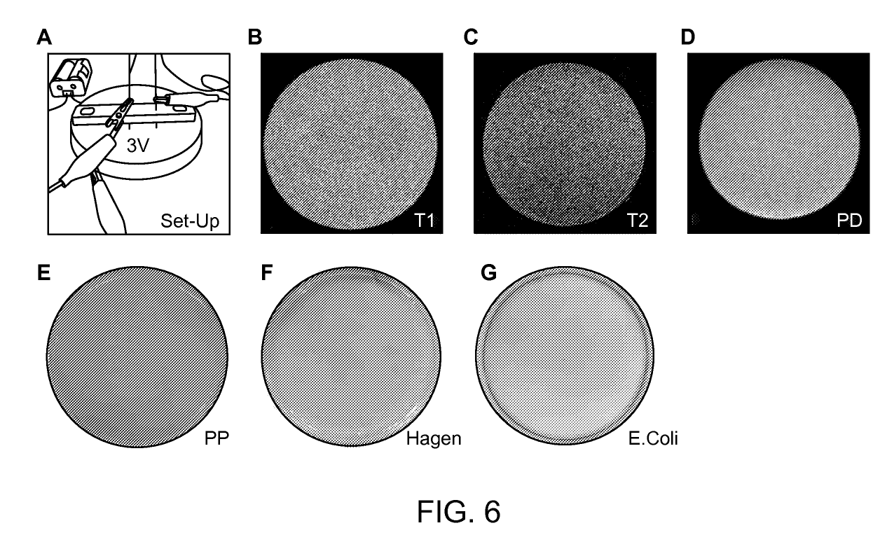

The experimental setup is shown in FIG. 6a. We have used two disposable graphite electrodes made of pencil lead (Pentel super HB 0.7 mm), to avoid contamination with products of electrolysis from the electrodes. The electrodes, mounted in a horizontal holder were placed perpendicularly to the gel phantom in the Petri dish. The electrodes were inserted 7 mm deep into the gel at a distance of 2 cm between their centers. The electrodes are connected to constant voltage batteries. We used 3V, 6V and 9 V batteries. The electrolysis process lasted 15 minutes. While typical electrolysis stimulation is administered using a fixed current source, we have used a fixed voltage source and have taken current measurements during the procedure (data not shown) for charge dosage estimation purposes. The overall delivered charge dosages over the 15 minutes stimulation period were approximately 0.9 C, 1.8 C and 2.9 C, for 3V, 6V and 9V, respectively. These charge dosages fall within the range of a typical electrolytic ablation therapy stimulation^{3, 38}. Identical experiments were done separately for MRI imaging, pH dyes based optical imaging and the E. coli viability model.

C. MRI Experimental Model

The phantom models were scanned, before and after electrolysis in a clinical 1.5 T MRI system (Philips Achieva SE) using a SENSE pediatric coil. The specific MRI parameters of each sequence are listed in Table 1. The mean acquisition time for each sequence was 3 minutes. Images shown in the paper were taken in the following order: T1W, T2W and PD. For later comparison with the MR images produced after electrolysis, FIGS. 6b, 6c and 6d show the baseline images for the sequences introduced above, respectively. In repeated experiments (results not shown) we changed the order and the time after the end of electrolysis at which the various sequences were taken and found no measurable effect of the time at which the images were acquired on the dimensions of the affected region.

D. pH-Sensitive Dye Model

As a control study, we used 2 distinct, pH sensitive dyes in order to estimate the boundary of the region where the pH has changed due to electrolysis. The dyes employed were 1) Phenolphtalein 1% by Sigma (turns pink in pH range of 8.2-12) and 2) Nutrafin pH wide range by Hagen (indicates pH by color in the range of 4.5-9). The dye manufacturer instructions prescribed the concentration of the last dye. We used a digital camera (Casio Exilim EX-ZR100) to acquire optical images of the experimental chamber before and after electrolysis and correlate these images with the images acquired using MRI and bacterial viability. For later comparison with images produced after electrolysis, FIGS. 6e and 6f, show the baseline images prior to electrolysis for the two dyes used, respectively.

E. Bacterial Model

Lyophilized E. coli of HB101 strain (BioRad) were grown in LB broth overnight and plated on LB broth based agar gel filled petri dishes. The LB broth for the overnight growth consisted of 1% BactoTryptone (BD), 0.5% Yeast Extract (BD), 1% NaCl (Sigma Aldrich) and 1.5% Agarose (Sigma Aldrich). 6 mm glass beads (Sigma Aldrich) were used for plating to ensure uniform coverage. After plating, the beads were removed and the plates were incubated for 15 minutes at 37°. The conductivity of the gel was measured around 0.2 S/m. After electrolysis the Petri dishes were covered and incubated for 24 hours. We used a digital camera (Casio Exilim EX-ZR100) to acquire optical images of the areas where bacterial growth was inhibited and correlated these images with the images acquired using MRI and gels with pH dyes. For later comparison with images produced after electrolysis, FIG. 6g shows the baseline images for an untreated with electrolysis, cell growth plate.

### Magnetic Resonance Imaging of Electrolysis

During electrolysis, pH fronts caused by evolution of protons (H⁺) and hydroxide (OH⁻) ions at the electrodes diffuse from the electrodes outward^{5, 23}. Fundamental studies on tissue ablation by electrolysis have shown that pH changes are indicative of electrolytic tissue ablation¹⁹. It was found that pH dyes marked gels²³⁻²⁵, could be used to study, monitor and image electrolysis.

Magnetic Resonance Imaging (MRI) has been used to study pH changes in biomedical settings with various methods and for various applications. For example, the effect of intracellular pH, as well as blood and tissue oxygen tension on T1 relaxation in the rat brain has been studied²⁷. Measurements of pH changes due to ischemia in the brain, in relation to amine and amide protons have been reported^{28, 29}. Measurements of pH changes due to kidney failure with an MRI-CEST pH responsive contrast agent, Iopamidol have been presented³⁰. A range of MRI-active pH indicators for food applications has been evaulated^{31, 32}. It has been shown that calf muscle T2 changes correlate with pH, PCr recovery and oxidative phosphorylation³³. Schilling et al. found that changes in intracellular pH affect the relaxation time of T2 in brain tissue³⁴. While reports exist on the use of MRI for detecting changes in pH, not until the present study has MRI been used to monitor the development of pH fronts during electrolysis. This study confirmed our hypothesis that pH fronts produced by electrolysis can be detected with MRI, and this approach may be applied to monitor cell ablation during electrolysis. Various approaches may be used to detect pH changes in tissue with MRI. We chose to explore our hypothesis with basic T1 weighted and T2 weighted based sequences for water.

An experimental study was conducted using a pH dye-stained physiological saline agar-gel based phantom as a model for a living tissue from an electrochemical standpoint. In the study, images obtained with MRI were compared to optical images acquired using pH-sensitive dyes. The MRI imaging sequences used were T1 weighted (T1W), T2 weighted (T2W) and Proton Density (PD). The optical images were acquired using pH-sensitive dyes embedded in the agar phantom exposed to electrolysis. In addition to validating the MRI-based approach using pH-sensitive dyes, we demonstrate a biological application of the MRI-based approach by comparing a spatial map of bacterial viability exposed to electrolysis with the MRI image of the phantom during electrolytic treatment.

### MRI Experimental Results

The agar plates were scanned before the administration of electrolytic treatment with the following sequences: T1W, T2W and PD. The gel plates were then electrolytically treated for 15 minutes using three different voltages: 3V, 6V, and 9V. After administering the treatment, the plates were immediately positioned in a pediatric head coil, and inserted into the MR scanner. MR sequences with the same pre-treatment parameters were then acquired. The MRI parameters are presented in Table 1.

TABLE 1
MRI Parameters

| | | | | | | | # of | Recon. |
|---|---|---|---|---|---|---|---|---|
| | | TR | TE | FOV | Slice | Num. | excitations | Matrix |
| Sequence | Coil | [ms] | [ms] | [mm] | thickness | slices | NSA | size |
| T1W | SENSE-Pediatric | 500 | 17 | 120 | 2 mm | 4 | 2 | 512 |
| TSE | Head coil | | | | | | | |
| T2W | SENSE-PED- | 1000 | 100 | 120 | 2 mm | 4 | 2 | 512 |
| TSE | HEAD | | | | | | | |
| PD | SENSE-PED- | 5000 | 30 | 120 | 2 mm | 4 | 2 | 512 |
| | HEAD | | | | | | | |

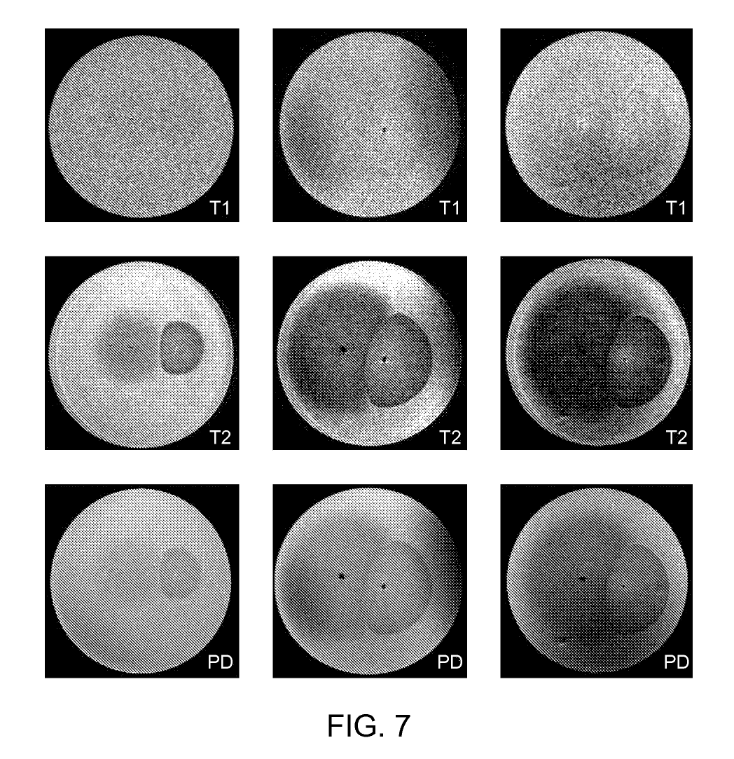

To facilitate comparison of results, FIG. 7 brings together images obtained for the three voltages and the three MRI sequences. The three columns are for the voltages of 3V, 6V and 9V, from left to right, respectively. The rows from top to bottom are for the following sequences: T1W, T2W, and PD, respectively. All the images are for a standard Petri dish with the same diameter, 8.5 cm. The electrolysis was administered via the same device, positioned at the same place for all the experiments, as constrained by the application rig in FIG. 6a. The position of the electrodes can be seen in some images as the two black traces (void of signal) at the centerline of the Petri dish. The distance between the electrodes was 2 cm. In all the images the anode is on the left and the cathode is on the right.

The first row of FIG. 7 shows images taken with the T1W sequence. The signal from the treated volume is iso-intense to hypo-intense. It is iso-intense for the lower voltage of 3V and becomes slightly hypo-intense with the increasing applied voltage. The pH change front is barely distinguishable for the 9V treatment. The second row shows results obtained with the T2W sequence. The margin of the electrolysis-affected region is marked with dotted yellow line. Hypo-intense signal can be seen in the treated region, with the signal intensity decreasing with increasing voltage. The affected region near the anode is larger than near the cathode. The altered pH front appears diffused in the anode-affected region and well-delineated in the cathode affected region. The interface between the cathode affected region and the anode affected region is distinct and visible. It is also note-worthy that in the cathode-affected region, the intensity adjacent to the cathode decreases with increasing voltage. The images produced with PD sequences, presented in the third row, show a generally similar pattern to that described for the T2W sequence produced images. Images produced with the PD sequence show a hypo-intense signal with lower intensity relative to the T2W sequence produced images.

### pH Dye Experiment

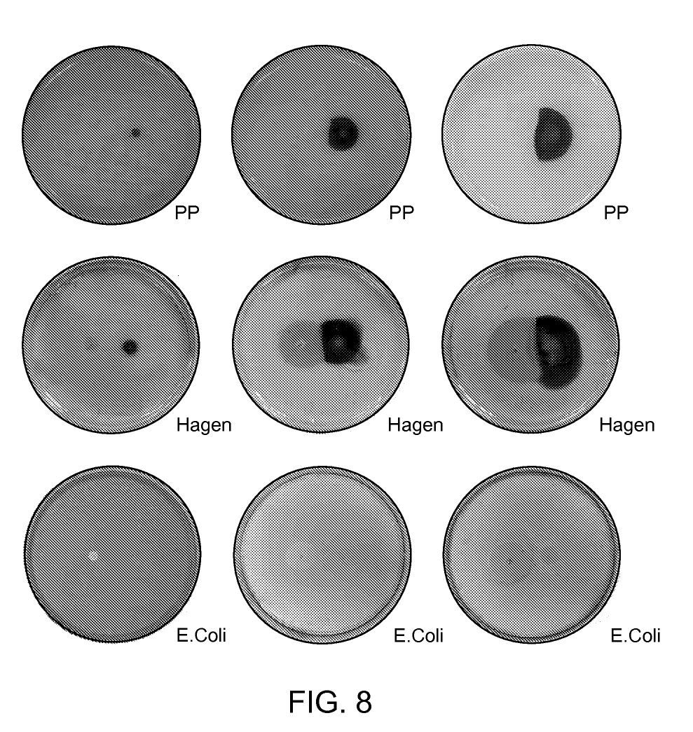

For the pH dye experiments we have infused the agar gel phantom described in our methods with two pH sensitive indicator dyes. FIG. 8 shows results obtained from the pH dye experiments. To facilitate the comparison of results, FIG. 8 brings together images obtained for the three voltages and results from the two-pH dyes infused gels. The three columns are for voltages of 3V, 6V and 9V, from left to right, respectively. The first row shows results obtained with phenolphthalein staining. The phenolphthalein stain produces a distinct pink color in the pH range from 8.2 to 12. The first row shows, as expected, an impression in only the cathode region on the right. The margins of the marked regions indicate a minimal pH of 8.2. For a voltage of 3V, the change of pH front takes a circular shape, most likely of a pH of 8.2. Increasing the voltage increases the size of the change in pH-affected area. Similar to the MRI images, the cathode-affected front collides with the anode produced front at a line between the electrode and cathode. The outer margin of the lesion that has a circular shape is most likely at a pH of 8.2, while the central line could be at any pH in the range of pH 8.2 to pH 12. It is interesting to note that immediately near the electrode for the 9V voltage the intensity of the image is reduced compared to a region further away from the electrode.

The second row shows the results of pH staining using the Hagen wide range pH testing kit. The cathodic region on the left is marked with a distinct blue color which indicates a basic pH in the vicinity of 8.3, while the anodic region on the right is marked with pink color which corresponds to pH level of 6.4. We have used the color-matching card provided by the manufacturer to establish the pH ranges. For 3V, the pH change affected regions have a circular shape. Increasing the voltage increases the size of the affected region. Just as for the other pH dye, and MRI images, the larger voltages lead to colliding pH fronts, which can observed as a straight line. Several interesting phenomena can be seen in the Hagen stained samples. First, for voltages of 6V and 9V, the areas immediately adjacent to the electrodes appear discolored relative to the surrounding stained areas. Furthermore, on the cathode side at 6V and 9V there is a drop of fluid, which was observed on the top of the gel. For 9V, some of the dye has leaked into this drop and stained it.

### Bacterial Viability Experiment

To demonstrate the relevance of our work to a biological model, electrolytic stimulation was applied to an agar dish plated with E. coli bacteria. The third row in FIG. 8 shows optical images of a bacterial viability pattern after treatment with 3V, 6V and 9V for 15 minutes, captured using a digital camera after 24 hour growth period. The anodic region on the right is marked with a clear bactericidal region increasing in area with increasing voltage. The cathodic region on the left is significantly smaller in terms of bactericidal area and is barely observable in the 3V image.

### Discussion

As can be observed in the T1W images (FIG. 7 first row), the treated volume exhibits hypointense to isointense signal, which indicates that the effect of electrolysis is minimal on T1W signal. A T1-weighted sequence produces an image where the signal contrast is determined by the differences in T1 relaxation times. The tissue signal in a T1 weighted imaging mode is inversely proportional to its T1 relaxation time. A short echo time (TE) is used to minimize T2-weighting together with a short repetition time (TR). A T1-weighted image is typically characterized by dark fluid signal due to the long T1 relaxation time of water. This result is consistent with previous studies of proton relaxation times in water as a function of pH²⁶ and show that T1 in water does not change in the range of from pH 2 to pH 12.

Visible changes are produced by electrolysis in T2 weighted images (FIG. 7, second row). In the T2-weighted imaging mode, the signal contrast is determined by differences in T2 relaxation times. The tissue signal in a T2 weighted image is proportional to its T2 relaxation time. A long repetition time (TR) is used to minimize T1-weighting together with a long echo time. The results in FIG. 7 are also consistent with previous findings showing that T2 in water is strongly affected by changes in pH and it increases symmetrically around pH 7 with an increase and decrease in pH.²⁶ Shilling et al.³⁴ provide an explanation for observed changes in T2 with changes in pH in the brain (which is consistent with the findings of Meiboom et. al.²⁶) by noting that the effect of pH on spin-spin relaxation time (T2) might be) explained by the fact that at pH 7.0, i.e., in the neutral environment, water molecules build larger hydrogen-bound mediated clusters than in the acid or base ranges. The reduced mobility leads to a prolonged correlation time for the dipolar interactions, which leads to a shortening of T2.³⁵ FIG. 7 shows that the electrolysis affected region near the anode is larger than that near the cathode. This difference makes physical sense and can be attributed to the relative radii of protons (H⁺, 0.88 fm) and hydroxide ions (OH⁻, 110 pm). Due to their relative smaller size, the protons are more mobile hence contributing to a larger extent to the conductivity increase around the anode. The increased mobility causes the pH region around the anode to be larger than around the cathode.

Proton Density (PD) is defined as the number of proton spins per unit volume of a tissue. Proton density may differ from the true water content due to short T2 components, which are not seen in MRI. So PD-weighted imaging where the T1 and T2 effects are minimized leads to images whose contrast is determined primarily by the spin (proton) density. This requires a short TE and long TR. In FIG. 7, the third row shows the process of electrolysis generated PD MRI images, which correspond well with the T2W images. This confirms that the observed images are related to the electrolysis caused diffusion of protons and hydroxide ions.

In FIG. 8, the first and the second rows show the results of the process of electrolysis obtained with pH stained dyes. While the optical pH results cannot be quantitatively compared with the MR images, because the pH dyes have a restricted range, both the MRI and pH dyes images show similar phenomena and trends. The observed affected zone increase in both modalities with an increase in voltage, which is consistent with the increased production of electrolytic compounds with voltage. The anode and cathode electrolysis affected regions meet at the same location in both the pH dye images and the MRI images. The pH dye results, in particular the second row of FIG. 8, show some additional interesting physical phenomena. The effect relates to the observed drops of water on the surface of the gel, during electrolysis with 6V and 9V. It is known that in an electric field, water moves by electro-osmosis from the anode to the cathode⁶. Therefore, during electrolysis, the gel near the anode tends to dehydrate while water accumulates near the cathode. This is the source of the water observed in the second row of FIG. 8. This electro-osmotic migration of water may be responsible for the discoloration adjacent to the cathode observed with both MRI and pH dyes.

FIG. 8 (row three) shows viability results from electrolysis treated E. Coli, grown on the surface of the gel. This part of the work is clinically relevant because electrolysis is becoming an important method for sterilizing surfaces and wounds, considering the growing antibiotic resistances of microorganisms³⁶. The pattern of cell ablation observed here is consistent with electrolytic ablation and further supports the idea that the MRI detected changes are relevant to electrolysis. FIG. 8 (row three) shows that the extent of cell ablation increases with the voltage and charge delivered as expected from a pH ablation process driven by electrolysis. It is also well established that the electrolytic products of the anode are more effective at cell ablation than the products of the cathode⁶. This is also confirmed in this study, which shows a much larger ablation zone near the anode than near the cathode.

A comparison between MRI images, pH dye based images and bacterial viability data shows that all the experiments, produce qualitatively similar results with respect to the effect of voltage on the affected area and with respect to the difference between the anodic and cathodic regions. A quantitative comparison is not possible because the pH dyes and the bacterial viability images represent limited ranges of pH. However, the results from the different imaging techniques show that increasing the voltage (charge delivered) increases the affected area in both the anode and cathode affected volume, the anodic front advances faster than the cathodic front, and the anode and cathode affected regions meet on the line perpendicular to the line connecting the electrodes.

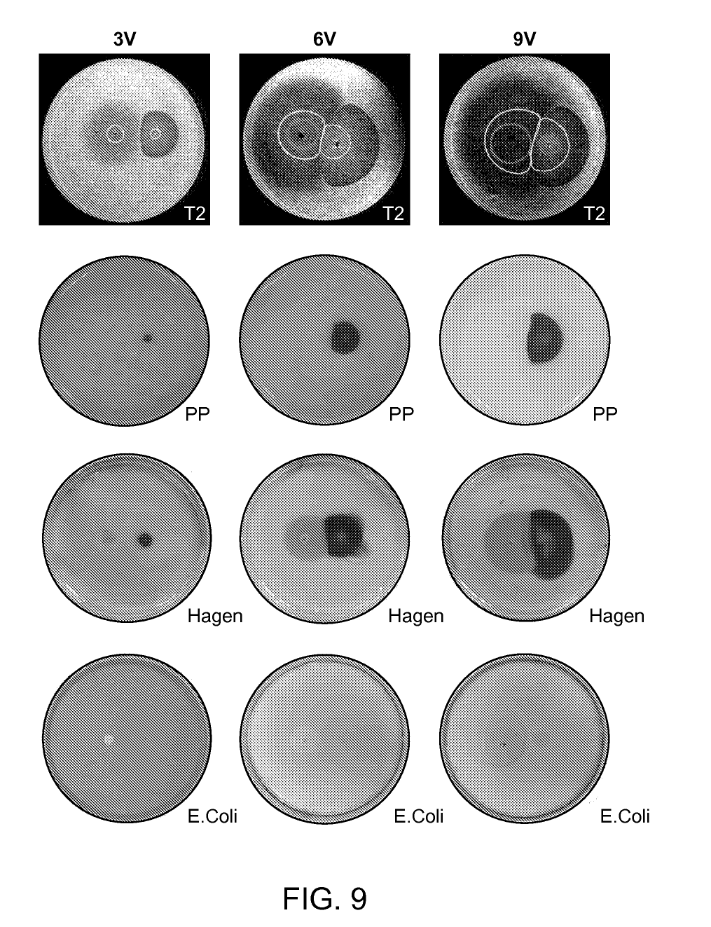

FIG. 9 summarizes the results. The first row shows the T2W MR image onto which we have superimposed the outline of the pH dye image (rows two and three) and the outline of the viability experiment (row four). It is interesting that the interface between the anode and the cathode affected zones lie on the same line in the MRI image and the pH dye image—suggesting that they both represent the same phenomenon. The overall shape of the pH dye image is similar to the MRI image. The affected zone observed with MRI is larger than that observed with dyes, because the range of changes that can be observed with MRI is not restricted by a certain pH dye value. The extent of cell ablation is substantially less than the extent of the region in which MRI detects changes in pH. In the past, studies on the effect of electrolysis on cell death were carried out using pH probes or pH dyes. This study demonstrates for the first time that MRI may be used in fundamental research on the effect of electrolysis on cells, as well as in a clinical setting to monitor therapeutic tissue ablation by electrolysis.

### Conclusion

The present study demonstrates that electrolysis-induced pH changes can be detected with MRI. The results indicate the feasibility of using MRI as a means to monitor dynamic changes in local pH level of a biological sample during an electrolysis process. This work used an agar-based gel model with conductivity in the range of a biological tissue, and is validated vs. optical images utilizing pH indicator dyes. In addition we demonstrate the relevance this approach in the biological context by correlating bacterial viability data with MRI measurements. It may be interesting to work on developing different MRI techniques for detecting pH, using MRI markers. It should be also possible to develop MRI sequences that detect discretely various ranges of pH, because T2 seems to be very sensitive to pH. This study demonstrates for the first time that MRI may be used in fundamental research on the effect of electrolysis on cells, as well as in a clinical setting to monitor therapeutic tissue ablation by electrolysis.
1. Amory, R. A treatise on electrolysis and its therapeutical and surgical treatement in disease. (William Woof &Co., New York; 1886).
2. Nordenstrom, B. E. W. Preliminary clinical trials of electrophoretic ionization in the treatement of malignant tumors. IRCS Medical Sc. 6, 537 (1978).
3. Nordenstrom, B. E. Electrochemical treatment of cancer. I: Variable response to anodic and cathodic fields. American journal of clinical oncology 12, 530-536 (1989).
4. Nilsson, E., Berendson, J. & Fontes, E. Development of a dosage method for electrochemical treatment of tumours: a simplified mathematical model. Bioelectrochemistry and Bioenergetics 47, 11-18 (1998).
5. Nilsson, E., Berendson, J. & Fontes, E. Electrochemical treatment of tumours: a simplified mathematical model. Journal of Electroanalytical Chemistry 460, 88-99 (1999).
6. Nilsson, E. et al. Electrochemical treatment of tumours. Bioelectrochemistry 51, 1-11 (2000).
7. von Euler, H., Nilsson, E., Olsson, J. M. & Lagerstedt, A. S. Electrochemical treatment (EchT) effects in rat mammary and liver tissue. In vivo optimizing of a dose-planning model for EChT of tumours. Bioelectrochemistry 54, 117-124 (2001).
8. von Euler, H., Nilsson, E., Lagerstedt, A. S. & Olsson, J. M. Development of a dose-planning method for electrochemical treatment of tumors: A study of mammary tissue in healthy female CD rats. Electro-and Magnetobiology 18, 93-+ (1999).
9. Bergues Pupo, A. E., Bory Reyes, J., Bergues Cabrales, L. E. & Bergues Cabrales, J. M. Analytical and numerical solutions of the potential and electric field generated by different electrode arrays in a tumor tissue under electrotherapy. Biomedical Engineering Online 10 (2011).
10. Placeres Jimenez, R. et al. 3D Stationary Electric Current Density in a Spherical Tumor Treated With Low Direct Current: An Analytical Solution. Bioelectromagnetics 32, 120-130 (2011).
11. Turjanski, P. et al. pH front tracking in the electrochemical treatment (EChT) of tumors: Experiments and simulations. Electrochimica Acta 54, 6199-6206 (2009).
12. Camue Ciria, H. M. et al. Antitumor effects of electrochemical treatment. Chinese Journal of Cancer Research 25, 223-234 (2013).
13. Yoon, D.-S. et al. Introduction of electrochemical therapy (EChT) and application of EChT to the breast tumor. Journal of Breast Cancer 10, 162-168 (2007).
14. Czymek, R. et al. Electrochemical Treatment: An Investigation of Dose-Response Relationships Using an Isolated Liver Perfusion Model. Saudi Journal of Gastroenterology 17, 335-342 (2011).
15. Griffin, D., Dodd, N., Zhao, S., Pullan, B. & Moore, J. V. Low-level direct electrical current therapy for hepatic metastases. I. Preclinical studies on normal liver. British journal of cancer 72, 31-34 (1995).
16. Griffin, D., Dodd, N.J., Moore, J. V., Pullan, B. & Taylor, T. The effects of low-level direct current therapy on a preclinical mammary carcinoma: tumour regression and systemic biochemical sequelae. British journal of cancer 69, 875-878 (1994).
17. Miklav\check{c}i\check{c}, D. et al. Tumor treatment by direct electric current-tumor temperature and pH, electrode material and configuration. Bioelectrochemistry and bioenergetics 30, 209-220 (1993).
18. Miklav\check{c}i\check{c}, D., Fajgelj, A. & Ser\check{s}a, G. Tumour treatment by direct electric current: electrode material deposition. Bioelectrochemistry and Bioenergetics 35, 93-97 (1994).
19. Finch, J. G. et al. Liver electrolysis: pH can reliably monitor the extent of hepatic ablation in pigs. Clinical Science 102, 389-395 (2002).
20. Olaiz, N., Suarez, C., Risk, M., Molina, F. & Marshall, G. Tracking protein electrodenaturation fronts in the electrochemical treatment of tumors. Electrochemistry Communications 12, 94-97 (2010).
21. Ivic, M. L. A., Perovic, S. D., Zivkovic, P. M., Nikolic, N. D. & Popov, K. I. An electrochemical illustration of the mathematical modelling of chlorine impact and acidification in electrochemical tumour treatment and its application on an agar-agar gel system. Journal of Electroanalytical Chemistry 549, 129-135 (2003).
22. Chu, K. F. & Dupuy, D. E. Thermal ablation of tumours: biological mechanisms and avdances in therapy. Nature Reviews/Cancer 14, 199-208 (2014).
23. Turjanski, P. et al. pH front tracking in the electrochemical treatment (EChT) of tumors: Experiments and simulations. Electrochimica Acta 54, 6199-6206 (2009).
24. Turjanski, P. et al. The role of pH fronts in reversible electroporation. PloS one 6, e17303 (2011).
25. Maglietti, F. et al. The Role of Ph Fronts in Tissue Electroporation Based Treatments. PloS one 8, e80167 (2013).
26. Meiboom, S., Luz, Z. & Gill, D. PROTON RELAXATION IN WATER. Journal of Chemical Physics 27, 1411-1412 (1957).
27. Kettunen, M. I., Grohn, O. H. J., Silvennoinen, M. J., Penttonen, M. & Kauppinen, R. A. Effects of intracellular pH, blood, and tissue oxygen tension on T-1 rho relaxation in rat brain. Magnetic Resonance in Medicine 48, 470-477 (2002).
28. McVicar, N. et al. Quantitative tissue pH measurement during cerebral ischemia using amine and amide concentration-independent detection (AACID) with MRI. Journal of Cerebral Blood Flow and Metabolism 34, 690-698 (2014).
29. Zong, X., Wang, P., Kim, S. G. & Jin, T. Sensitivity and Source of Amine-Proton Exchange and Amide-Proton Transfer Magnetic Resonance Imaging in Cerebral Ischemia. Magnetic Resonance in Medicine 71, 118-132 (2014).
30. Longo, D. L., Busato, A., Lanzardo, S., Antico, F. & Aime, S. Imaging the pH evolution of an acute kidney injury model by means of iopamidol, a MRI-CEST pH-responsive contrast agent. Magnetic Resonance in Medicine 70, 859-864 (2013).
31. Evans, S. & Hall, L. Evaluation of a range of MRI-active pH indicators using a multiple-sample method. Aiche Journal 51, 1541-1547 (2005).
32. Evans, S. & Hall, L. Measurement of pH in food systems by magnetic resonance imaging. Canadian Journal of Chemical Engineering 83, 73-77 (2005).
33. Schmid, A. I. et al. Exercising calf muscle T-2 changes correlate with pH, PCr recovery and maximum oxidative phosphorylation. Nmr in Biomedicine 27, 553-560 (2014).
34. Schilling, A. M., Blankenburg, F. B., Bernarding, J., Heidenreich, J. O. & Wolf, K. J. Intracerebral pH affects the T2 relaxation time of brain tissue. Neuroradiology 44, 968-972 (2002).
35. Schilling, A., Blankenburg, F., Bernarding, J., Heidenreich, J. & Wolf, K. Intracerebral pH affects the T2 relaxation time of brain tissue. Neuroradiology 44, 968-972 (2002).
36. Piggott, J. M., Berney, H., Hurley, E. & Clair, J. Planar flexible electrode for use in wound sterilization. Conference Proceedings. Second Joint EMBS-BMES Conference 2002. 24th Annual International Conference of the Engineering in Medicine and Biology Society. Annual Fall Meeting of the Biomedical Engineering Society (Cat. No. 02CH37392), 1929-1930 vol. 1923 (2002).
37. Laufer, S., Ivorra, A., Reuter, V. E., Rubinsky, B. & Solomon, S. B. Electrical impedance characterization of normal and cancerous human hepatic tissue. Physiological measurement 31, 995 (2010).
38. Nordenström, B. Biologically closed electric circuits: Clinical, experimental and theoretical evidence for an additional circulatory system. (Ursus Medical AB, 1983).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

What is claimed is:

1. A method comprising:
monitoring electrolysis of a tissue in a subject using an imaging technique selected from the group consisting of:
electrical impedance-based imaging, wherein the method comprises:
imaging the electrical impedance of a tissue of the subject undergoing electrolysis; and
monitoring the electrolysis based on one or more electrical impedance images of the tissue, and
magnetic resonance imaging (MRI), wherein the method comprises:
imaging pH changes in a tissue of the subject undergoing electrolysis by magnetic resonance imaging; and
monitoring the electrolysis based on one or more magnetic resonance images of the pH changes in the tissue.

2. The method according to claim 1, wherein the imaging technique is electrical impedance-based imaging.

3. The method according to claim 2, wherein the electrical impedance-based imaging comprises electrical impedance tomography (EIT).

4. The method according to claim 3, wherein the imaging comprises magnetic electrical impedance tomography (MEIT).

5. The method according to claim 1, wherein the imaging technique is magnetic resonance imaging (MRI).

6. The method according to claim 5, wherein imaging pH changes in the tissue of the subject undergoing electrolysis comprises imaging pH fronts in the tissue undergoing electrolysis.

7. The method according to claim 5 or claim 6, wherein the magnetic resonance images are produced using a sequence selected from: a T1 weighted sequence, a T2 weighted sequence, a proton density (PD)-weighted sequence, and combinations thereof.

8. The method according to any one of claims 1 to 7, wherein the method further comprises comparing the one or more electrical impedance images or magnetic resonance images to a reference image.

9. The method according to claim 8, wherein the reference image is an electrical impedance image or magnetic resonance image of the tissue obtained at an earlier time.

10. The method according to claim 9, wherein the method comprises:
imaging the electrical impedance or pH changes of the tissue at a second time point;
comparing the electrical impedance image or magnetic resonance image of the pH changes in the tissue at a first time point to the electrical impedance image or magnetic resonance image of the pH changes in the tissue at the second time point; and
monitoring the electrolysis based on the comparison.

11. The method according to claim 10, wherein the first time point is prior to the start of electrolysis, and the second time point is after the start of electrolysis.

12. The method according to claim 10, wherein the first time point is after the start of electrolysis, and the second time point is after the first time point.

13. The method according to any one of claims 1 to 12, wherein the electrolysis is cathode-based.

14. The method according to any one of claims 1 to 13, wherein the tissue is selected from the group consisting of: brain tissue, lung tissue, heart tissue, muscle tissue, skin tissue, kidney tissue, cornea tissue, liver tissue, abdomen tissue, head tissue, leg tissue, arm tissue, pelvis tissue, chest tissue, prostate tissue, breast tissue, esophagus tissue, GI tract tissue and trunk tissue.

15. The method according to any one of claims 1 to 14, wherein the electrolysis is electrolytic surgery to ablate tissue.

16. The method according to claim 15, wherein the tissue to be ablated is a soft tissue neoplasm.

17. The method according to any one of claims 1 to 13, wherein the electrolysis is electrolytic surgery to treat an ischemic disease of the eye.

18. The method according to any one of claims 1 to 13, wherein the electrolysis is electrolytic surgery to promote wound repair.

19. A system for performing tissue electrolysis in an individual, the system comprising:
an electrolytic device, and
an electrical impedance measuring device.

20. A system for performing tissue electrolysis in an individual, the system comprising:
an electrolytic device, and
a magnetic resonance imaging device.
